(19) United States
(12) Patent Application Publication
(10) Pub. No.: US 2026/0004928 A1
(43) Pub. Date: Jan. 1, 2026

(54) AUTONOMOUS PATIENT MONITORING WITH PROMPTED EVENT DETECTION IN A TELEHEALTH SYSTEM

(71) Applicant: TELADOC HEALTH, INC., Purchase, NY (US)

(72) Inventor: Paul C. McElroy, Goleta, CA (US)

(21) Appl. No.: 18/906,313

(22) Filed: Oct. 4, 2024

Related U.S. Application Data
(63) Continuation-in-part of application No. 18/758,998, filed on Jun. 28, 2024.
(60) Provisional application No. 63/671,250, filed on Jul. 14, 2024.

(51) Int. Cl. G16H 40/67; G16H 30/40; G16H 40/20

(52) U.S. Cl. CPC G16H 40/67; G16H 30/40; G16H 40/20

(57) ABSTRACT

Systems and methods for autonomous patient monitoring in a telehealth system. The system may include an in-room device including a camera and other devices to extract information from the patient environment in real-time. The information from the patient environment may be processed using object detection and other algorithms to identify and track relevant objects and/or events over time. The system may be configured to notify a care provider of specific events. A multi-modal large language model (MMLLM) may be employed to interpret the information about the patient environment and prompts or instructions from a care providers to monitor for and raise alerts or perform other actions in response to the detection of specific events. The system may also maintain a compact, text-based history of events in the patient environment that can be searched or interpreted by the MMLLM in response to user prompts.

### CROSS-REFERENCE TO RELATED APPLICATIONS

This application is a continuation-in-part of U.S. application Ser. No. 18/758,998, filed Jun. 28, 2024, and a claims priority to U.S. provisional application No. 63/671,250, filed Jul. 14, 2024, the contents of which are hereby incorporated by reference in their entirety.

### TECHNICAL FIELD

The present disclosure pertains to telehealth systems and more specifically to autonomous detection of patient status in a care environment.

### BACKGROUND

Telemedicine, telehealth, and/or virtual care is the provisioning of health care services using communications devices, such as personal computers (e.g., laptops, desktops, tablets, smartphones, etc.) and/or purpose-built devices (e.g., telemedicine carts, etc.) coupled to a communications network. Virtual care may involve a patient using a device to connect to and communicate with a remote health care provider, which may be a physician, clinician, counselor, coach, or trainer, to address health concerns of the patient. Virtual care may also be delivered in in-patient settings. In these cases, a remote provider may use a communication device to connect to another communication device located in a patient room, emergency room, operating room or other care location within a hospital or other healthcare facility. In-patient virtual care often involves a remote specialist consulting with local care providers, communicating with patients, and/or proctoring surgeries or other medical procedures.

Virtual care sessions may involve a two-way audiovisual conference between the remote provider and the patient and/or local provider, communication of medical data obtained from medical instruments coupled to a communication device, communication of health records between the local and remote sites, as well as communication of diagnoses, recommendations, and/or prescription information from the remote provider to the patient, local provider, and/or third parties such as electronic health records providers, insurance providers, and/or pharmacies.

Prior art telehealth devices in in-patient settings often take the form of wheeled carts equipped with video conferencing systems that may be moved from room to room. These carts may be moved by a bedside caregiver who also communicates and coordinates with a remote physician when the patient is available to participate in the telehealth consult. It is becoming increasingly common, however, for hospitals to equip patient rooms with dedicated in-room connected care devices. These are in-room, fixed telehealth devices that do not require a bedside caregiver to be in attendance with the patient or to move equipment around. These devices are often mounted to a wall and connected to a TV that is in the room so healthcare providers can virtually interact with a patient. Using these devices, a remote doctor, nurse, or other caregiver to connect to the telehealth device to conduct a 2-way audio/video session whenever the patient is available.

### BRIEF SUMMARY

The present disclosure includes a system and method for autonomous patient monitoring system and method with prompted event detection. The system leverages real-time video and other data captured by in-room connected care devices and multi-media large language models to enable a more intuitive and highly configurable means for detecting relevant events in a patient care setting.

One embodiment of the disclosure is a method for autonomous patient monitoring in a telehealth system. The method comprises displaying a user interface at a provider device. The user interface includes a care location menu, a video window, and a prompt editor. The care location menu includes a list including a plurality of remote care locations. The method includes receiving a selection of at least one care location from a user via a user input device and displaying video received from a camera installed at the selected care location in the video window. The method further includes receiving a prompt via the prompt editor. The prompt comprises a text string that includes a description of an event relating to the care location and an action to be performed when the event is detected. An application programmer interface (API) call is communicated to a multi-media large language model (MMLLM) via an MMLLM interface. The API call includes an instruction to notify a response handler when the event is detected in the video received from the care location. When the notification from the MMLLM interface is received at the response handler, the action included in the prompt is performed.

Another embodiment of the disclosure is system for autonomous patient monitoring in a telehealth system. The system comprises a controller. The controller displays a user interface at a provider device. The user interface includes a care location menu, a video window, and a prompt editor. The care location menu includes a list of a plurality of remote care locations. The controller is configured to receive a selection of at least one care location from a user via a user input device coupled to the provider device. The controller displays video received from a camera installed at the selected care location in the video window. The controller receives a prompt via the prompt editor. The prompt comprises a text string that includes a description of an event relating to the care location and an action to be performed when the event is detected. The controller communicates an application programmer interface (API) call to a multi-media large language model (MMLLM) via an MMLLM interface. The API call includes an instruction to notify a response handler when the event is detected in the video received from the care location. When the response handler receives the notification from the LLM, the controller performs the action included in the prompt.

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof which are illustrated in the appended drawings. Understanding that these drawings depict only example embodiments of the disclosure and are not therefore to be considered to be limiting of its scope, the principles herein are described and explained with additional specificity and detail through the use of the accompanying drawings in which:

FIG. 1 is a schematic diagram of a telehealth system according to one embodiment of the present disclosure.

FIG. 2 is a schematic diagram of a patient room telehealth device according to one embodiment of the present disclosure.

FIG. 3 is a schematic diagram of an autonomous patient monitoring module according to one embodiment of the present disclosure.

FIG. 4 is a flowchart of a method for autonomous patient monitoring according to one embodiment of the disclosure.

FIG. 5 illustrates an example of a user interface including a patient dashboard in an autonomous patient monitoring system according to one embodiment of the present disclosure.

FIG. 6 is a schematic diagram of an autonomous patient monitoring module with prompted event detection according to one embodiment of the present disclosure.

FIG. 7 is a flowchart illustrating a method for autonomous patient monitoring with prompted event detection according to one embodiment of the present disclosure.

FIG. 8 is an example of a user interface in a system for autonomous patient monitoring with prompted event detection according to one embodiment of the present disclosure.

FIG. 9 depicts an example computing system that may implement various systems and methods according to embodiments of the present disclosure.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without departing from the spirit and scope of the disclosure.

It should be understood at the outset that although illustrative implementations of one or more embodiments are illustrated below, the disclosed apparatus and methods may be implemented using any number of techniques. The disclosure should in no way be limited to the illustrative implementations, drawings, and techniques illustrated herein, but may be modified within the scope of the appended claims along with their full scope of equivalents.

A typical telehealth encounter may involve a patient and one or more remotely located physicians or healthcare providers. Devices located in the vicinity of the patient and the providers allow the patients and providers to communicate with each other using, for example, two-way audio and/or video conferencing.

A telepresence device may take the form of a desktop, laptop, tablet, smart phone, or any computing device equipped with hardware and software configured to capture, reproduce, transmit, and receive audio and/or video to or from another telepresence device across a communication network. Telepresence devices may also take the form of telepresence robots, carts, and/or other devices such as those marketed by Teladoc Health, Inc. of Purchase, New York, under the names VITA, LITE, VANTAGE, VICI, VIEWPOINT, XPRESS, and XPRESS CART. Telepresence devices may also take the form of stationary, in-room devices such as the Teladoc Health TV Pro 300, which may be installed in patient rooms and connected to existing audio/video hardware in the patient room, such as a TV or video display device. The physician telepresence device and the patient telepresence device may mediate an encounter, thus providing high-quality audio capture on both the provider-side and the patient-side of the interaction.

In addition to providing real-time communication capabilities in the virtual care context, an in-room telehealth device in accordance with the present disclosure may also provide continuous, autonomous patient monitoring, as described more fully below.

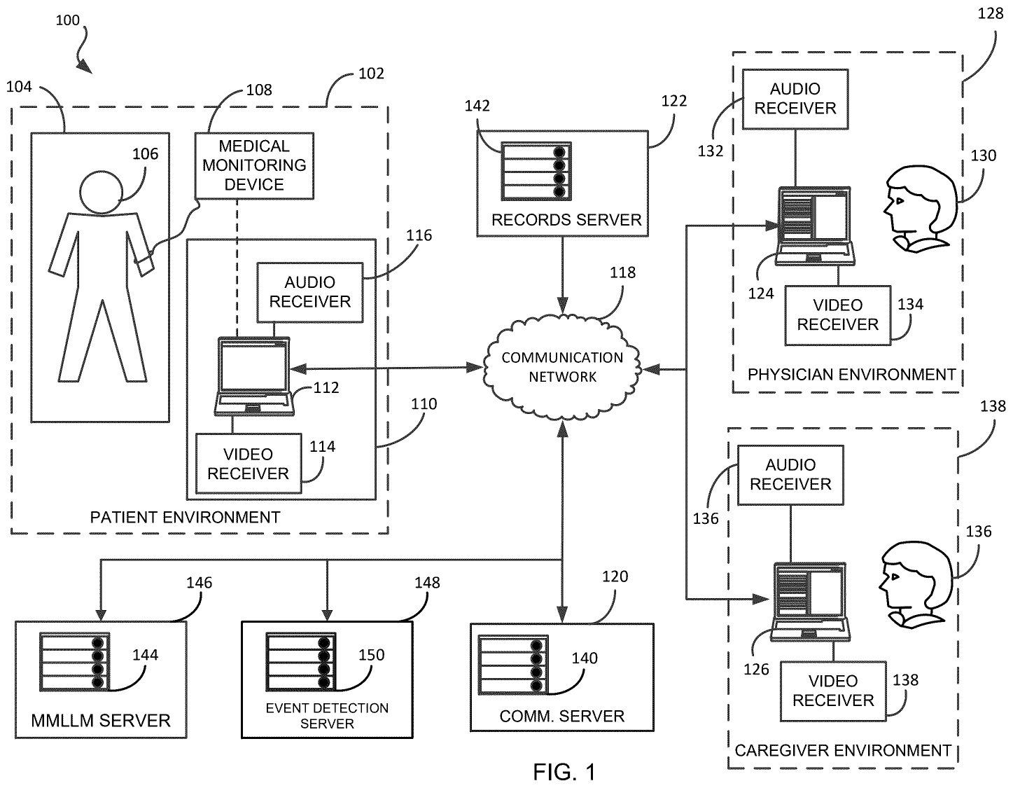

FIG. 1 illustrates a telehealth system 100 automatically determining the call availability status of a patient located in a patient environment 102, such as in-patient care facility. The patient environment 102 may include a patient bed 104, or other furniture, such as a chair, where the patient is situated when present in the room. The patient bed 104 may or may not have a patient 106 in it at any given time. The patient 106 may be connected to one or more medical monitoring devices 108 to monitor the patient's vital signs or other health indicators. The patient environment 102 may a hospital room, out-patient clinic room, nursing home room, a room in a patient's home, or any other location where a patient may receive care and/or be monitored.

The patient environment 102 may also include an in-room telehealth device 110, which may include at least a computing device 112, a video receiver 114 in the form of one or more cameras, and an audio receiver 116 in the form of one or more microphones. The computing device may take the form of any computing device capable of performing the automatic patient presence detection function described in detail below. Examples of computing devices include laptops, desktops, smart phones, as well as dedicated telehealth devices such as the TV PRO 300 marketed by Teladoc Health, Inc.

The system 100 may also include a communications network 118 that connects the telehealth device 110 to a communication server 120, a records server 122, a multi-modal large language model (“MMLLM”) server 144 a provider device 124, and a caregiver device 126.

The provider device 124 may be operated by a physician 130 located in a physician environment 128, such as a hospital, the physician's office, home, car, or any other location with network connectivity. The physician device may be any telepresence-capable device as discussed above and, like the telehealth device 110 in patient environment 102, include or be coupled to an audio receiver 132 and a video receiver 134.

The caregiver device 126 may be operated by a caregiver 136 in a caregiver environment 138. The caregiver 136 may be a nurse or other caregiver to the patient 106. The caregiver environment 138 may be a nurses' station within the facility that houses the patient environment 102 or any location including one or more caregivers tasked with monitoring the patient 106 and possibly other patients. Like the provider device 124, the caregiver device 126 may be any telepresence device as discussed above and, like the telehealth device 110 in patient environment 102, include or be coupled to an audio receiver 136 and a video receiver 138.

The communication server 120 may be one or more remotely connected computer servers 140 that provide various computing functions to support the functions of the telehealth system. The communication server may, for example, facilitate call setup, manage user authentication and permissions, monitor telehealth devices 110 and their statuses, report device status and usage information, deploy software and firm updates, as well as provide communications services such as firewall traversal and/or ICE/STUN/TURN protocols. In some examples, the communication server 120 may include a virtual server and the like provided over a cloud-based service, as will be understood by a person having ordinary skill in the art.

The large language module (“LLM”) server 144 may provide multi-modal large language model processing for the in-room device 110, caretaker device 126, remote provider device 124, or any device coupled to the network. The LLM server 144 can be a computer server 146 remotely connected to the in-room, caretaker, or provider devices via the communication network 118 or may be onsite with the physician 106, caretake 136, or the patient 106. The LLM server may 144 may be used in autonomous patient monitoring as described more fully below. Suitable LLMs for use in the disclosed system include multi-media LLMs such as ChatGPT 4o, Phi3.5-Vision, Llama 3.2, and Florence 2. Ideally, the LLM will be multi-modal, supporting both video and audio (though video is sufficient for much of the functionality described herein), and trained on a sufficiently large corpus of data to accurately identify and/or describe events in video.

Both the physician 130 and the caregiver 136 may retrieve and review an electronic medical record (“EMR”) and other medical data related to the patient 106 from a networked records server 122. The records server may receive medical data directly from the medical monitoring device 108. The records server 122 can be a computer server 142 remotely connected to the telehealth device 110, medical monitoring device 108, provider device 124 and/or the caregiver device 126 via the communication network 118 or may be onsite with the physician 106, caregiver 136, or the patient 106. Either the records server 122 or communication server 120 may also provide a scheduling service that allows the patient 106, provider 130, and/or caregiver 136 to schedule telehealth visits between patient 106 and provider 130. The schedule may be visible to one or more of the parties via a browser or dedicated app running on the local device. When the appointment nears, one or more of the parties may receive a reminder notification on their device. The reminder may include a link that the local user can activate to initiate the telehealth call.

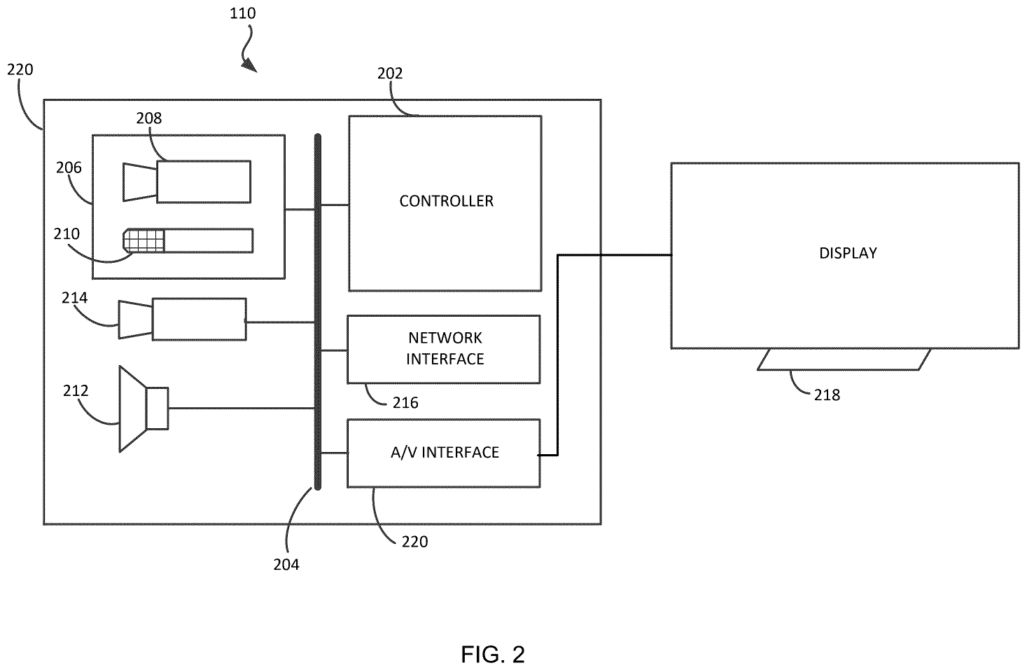

FIG. 2 is a schematic diagram of a telehealth device 110 in accordance with one embodiment of the present disclosure. The device 110 may include a housing 220 that contains a controller 202 coupled to a bus 204. The bus 204 may be coupled to a pan-tilt-zoom (“PTZ”) camera system 206 capable of moving a video camera 208 and a microphone 210 together around a pan axis and tilt axis. The PTZ system 206 may be remotely controlled by the remote provider during a telehealth consult to adjust the field of view of the camera 208 to provide an optimal view of the patient. The camera 208 may have a high optical zoom factor, e.g., 30×, that can be remotely controlled to retrieve high-resolution images of objects around the room, as well as to allow the provider to examine the patient's skin, eyes, etc., at a resolution that meets or exceeds that of an unassisted eye during an in-person visit. Microphone 210 is mounted on PTZ system 206 to face the same direction as the camera 208 and moves (e.g., pans and tilts) with the camera. Microphone 210 may be a directional microphone designed to isolate on-axis sound emanating from the direction the microphone is facing (i.e., the field of view of the camera) and suppresses off-axis sound emanating from elsewhere. This arrangement can produce a higher quality audio experience for the provider when listening to the patient during the telehealth consult.

The telehealth device 110 may also include a speaker 212 and a stationary camera 214 coupled to the bus 204. The speaker may reproduce sound captured by a microphone of the provider or caregiver devices during the telehealth consult, allowing the patient to hear the provider or caregiver. The stationary camera 214 may provide a wide field of view or “wide angle” that includes all or most of the patient room. The stationary camera 214 may also include an infrared filter that allows the caregiver to monitor the patient room when the room is dark.

The telehealth device 110 may include a network interface 216 that transmits audio, video, status, and other data from the other components of the telehealth device 110 to other elements of the telehealth system via the network. Network interface 216 may also receive audio, video, control, status, and other data from other components of the telehealth system via the network. Network interface 216 may include a wired and/or wireless connection to a local area network (LAN) that serves the patient environment. This LAN may provide access to the Internet via an Internet service provider. By way of example, network interface 216 may transmit video from camera 208 and audio from microphone 210 to the remote provider's device via the Internet. Likewise, the network interface 216 may receive audio, video, and control data from the provider device via the Internet. The network interface may also transmit status data from the controller to the communication server via the network.

The telehealth device 110 may be coupled to a TV or display device 218 via an A/V interface 220 coupled to the bus 204. A/V interface 220 may relay video data received from the provider device, via the network interface, to the display device 218 for display. By way of example, the display device 218 may display video of the provider's face for the patient to view during the telehealth consult.

The telehealth device 110 may include a power supply (not shown) coupled to a battery, power cord, or both. The power supply may be coupled to bus 204 and provide electrical power to the other components of the device 110 via the bus 204 or dedicated power connections.

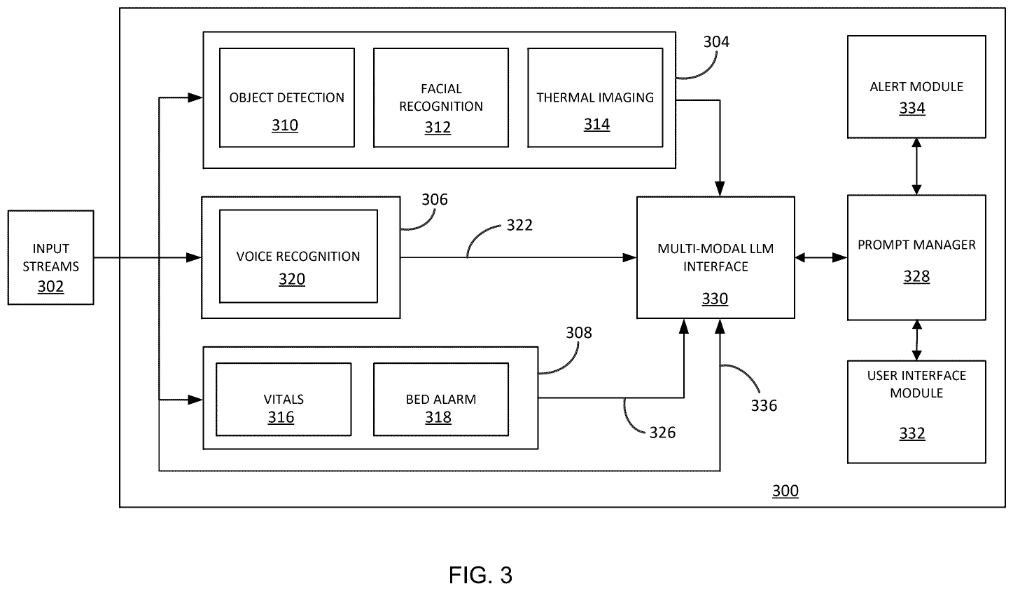

FIG. 3 is a schematic diagram of an autonomous patient monitoring module 300 that may be executed on the controller 202 of in-room telehealth device 110 (see FIG. 2), caretake device 126 (FIG. 1), provider device 124 (FIG. 1), or any other device connected to the network 118 (FIG. 1), such as the communications server 120 or event detection server 148 (FIG. 1). The module 300 may include several modules for analyzing different data types that may be available in the input streams 302, including an image analysis module 304, an audio analysis module 306, and a monitoring device module 308.

Input streams 302 may include video data, audio data, and/or or any other available data. Other data may include data available from a vital signs monitors, bed alarms or other medical monitoring device data. The input streams 302 may undergo one or more modes of analysis depending on the availability of each data type, user preferences, organizational policies, laws, regulations, and the like. For example, if only audio is present in the stream 302, the module 300 may only use the audio analysis module 306. If, however, the stream 302 contains both audio and video data, the module may employ both the image analysis module 304 and the audio analysis module 306, etc.

Within each module may be one or more algorithms that perform a mode of autonomous patient monitoring specific to that module. For example, image analysis module 304 may include one or more of an object detection algorithm 310, a facial recognition algorithm 312, and a thermal imaging algorithm 314. Similarly, the medical monitoring device module 308 may include algorithms that can monitor the patient based on vitals monitoring data 316 and or data from a patient bed alarm 318, which may use a weight sensor to detect whether the patient is currently occupying the bed. The audio analysis module 306 may include a voice recognition algorithm 320 that can recognize speech and other audio signatures that may be present in the patient room. Each algorithm may output information packets representing what the algorithm detects in the input streams.

The image analysis module 304 may include an object detection algorithm 310 that can identify objects or areas of interest within the video stream. Areas of interest may include the patient bed, chairs, tables, doorways, and medical equipment present in the patient room. An area of interest may also include a human being that is detected in the stream. The object detection algorithm 310 may output a table or vector that includes a list of objects detected in an image, a set of spatial coordinates that describing a bounding box encompassing each detected object within the image, and/or an image data representing each detected object.

The object detection may be performed using one or more machine learning algorithms that have been trained to identify standard objects. In addition, the object detection may also apply logic or rules specific to a hospital room and/or particular use cases. For example, the algorithm may be configured to detect one or more persons in the room and identify one of the persons as a patient by determining which person is in the hospital bed. The algorithm may also then continuously track that person's identity as the patient over time using a tracking algorithm. In one embodiment, the object detection algorithm may be implemented as a convolutional neural network such as YOLO (You Only Look Once). The tracking algorithm may be implemented as Vision Transform (ViT) model. The object detection and tracking functions may also be implemented using other suitable algorithms as will be known to those of skill in the art.

The image analysis module 304 may also employ a facial recognition algorithm 312 that can recognize faces within an image and even identify the person associated with a detected face where a database with information linking a specific set of facial features to the identity of the person is available. The facial recognition module 312 may be trained to identify the patient's face as well as the faces of hospital staff when the necessary training data is available (such as a database of photographs of patients and/or staff). In addition, the facial recognition algorithm may learn to recognize the face of the patient, as opposed to staff or other visitors, by noting the position and duration of the face relative to certain areas of interest. For example, the facial recognition algorithm may be configured to recognize a face detected at the head of the patient bed for long periods of time as the face of the current patient associated with the patient room.

Certain modes of analysis may not be performed even when the necessary data is available. For example, if patient preferences or organization policies prohibit the use of facial recognition technology, the facial recognition 312 would not be performed on any incoming video data.

In one embodiment, the system may employ a multi-model LLM for autonomous patient monitoring. In this embodiment, each of the modules 304, 306, 308 may output data from its respective algorithm(s) to a multi-modal LLM interface 330. The multi-modal LLM interface may also receive one or more of the available input streams, such as video, audio, etc., directly, as shown via arrow 336, and without having undergone any processing by the various modules 304, 306, and/or 308. The LLM interface 330 may provide access to a multi-modal modal LLM, which may be running locally on the in-room device or on a remote server, such as MMLLM server 146, or any other device coupled to the in-room device via a network. The LLM interface 330 may also be coupled to a prompt manager 328, which may manage the generation of prompts for the LLM and handle responses received from the LLM via the LLM interface 330. The connections and/or information flows between the various modules in FIG. 3 are illustrative of just one embodiment of the module 300. In other embodiments, for example, the user interface module 332 may communicate directly with the LLM interface 330 and/or alert module 334.

The prompt manager 328 may be configured to receive the input streams and the outputs of the various modules 304, 306, 308, either directly or via the LLM interface module 330. For example, the prompt manager may receive video patient environment as well as data representing features detected in that video by the various modules/algorithms. The prompt manager may, via the LLM interface 330, prompt the LLM to further analyze the detected features. By way of example, when the object detection algorithm outputs that it detected a vital signs monitor along with an image of the vital signs monitor, the prompt manager may automatically generate a prompt requesting the LLM to analyze the image of the vital signs monitor and return a table comprising a row including a label for each data field detected in the image of the vital signs monitor and the value of the corresponding field identified in the image.

The module 300 may also include a user-interface module 332 that communicates with the LLM interface 330. The UI module 332 may receive information from the LLM and format it for display on a user interface or dashboard presented to a user of the autonomous patient monitoring system. The module 300 may also include an alert module 334 that communicates with the LLM interface 330 and/or UI module 332. The alert module 334 may be configured to compare the values of certain information fields returned by the LLM to specified thresholds or ranges. When any particular value falls outside of its corresponding pre-specified range, the alert module 334 may generate an alert that is presented to one or more users of the system. The alert may take the form of visual or audible alerts presented at a device of a provider or caretake. The alerts may be communicated natively within a given application program running on the user device or converted to SMS text message or other communication protocols and transmitted to the phone or other device of designated providers, caretakes, or family members.

The module 300 may reside on a controller or computing device located elsewhere (e.g., communication server 120, FIG. 1, or any other suitable computing device in communication with the telehealth device 110). Images of the patient may, however, constitute personally identifiable information or protected health information, and thus certain laws and or organizational policies may prevent such images from being transmitted to another location without the patient's explicit consent, which may not always be possible to obtain. Thus, in a one embodiment of the invention, the autonomous patient monitoring is performed on the device located in the patient's room, thereby eliminating the need to transmit images of the patient over a network.

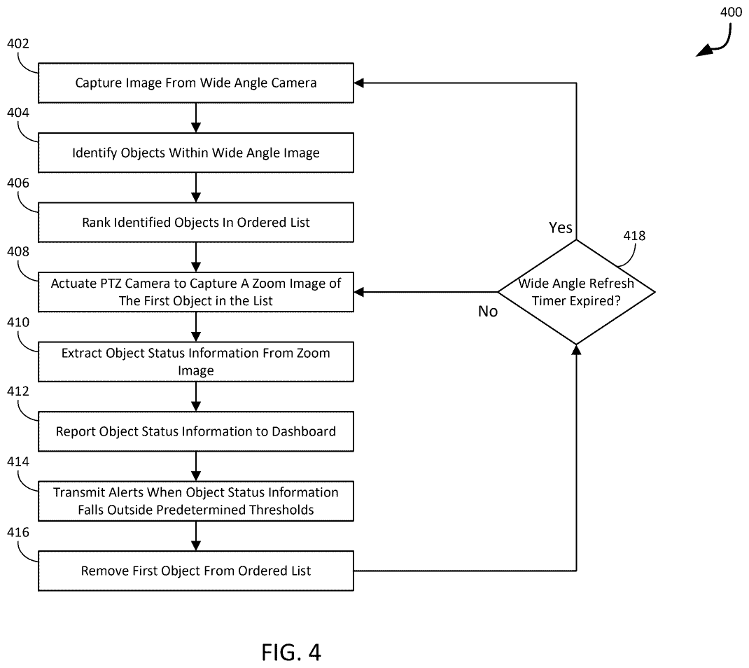

FIG. 4 is a flow chart illustrating a method of autonomous patient monitoring 400 in accordance with an embodiment of the disclosure. The method begins at step 402, in which the controller receives a video image from the stationary or wide-angle camera. At step 404, the wide angle image is analyzed using an object detection routine to identify relevant objects within the scene. The object detection routine may be a machine learning algorithm that has been trained to identify a specific set of objects including but not limited to humans, patient beds, intravenous (IV) fluid bags, IV pumps, IV tubing, IV ports attached to the patient, medical monitoring devices, medical monitoring sensors that may be attached (or not attached) to the patient, medical charts, medical imaging displays and other objects that may be present in a patient care environment. In addition to identify humans generally, where the object detection has access to a facial recognition database, the system may also identify specific humans in the image. This may be useful for identifying the patient and tracking the identity and schedule of people who have entered the patient room.

As there may be many identifiable objects in the wide angle image that are not relevant to monitoring a particular patient, or otherwise not of interest to the user, the object detection routine may be configured to identify a particular set of objects and ignore other objects. The system may include a user interface that displays a list of objects the controller is capable of identifying and allows a user to select and/or deselect one or more objects from the list for tracking. In addition, the system may include a user interface that allows the user to train the controller on new objects to be identified. By way of example, the system may enable the user to capture or upload one or more images containing a new object of interest, highlight regions within the images that contain the object of interest, and create a label for the object. The system may then add the newly defined object of interest to the list of objects the user can choose from for tracking.

The object detection routine may return, for each object it identifies in the image, a label representing the type of object that was detected and a vector that defines a bounding box. The bounding box may be the smallest rectangle that can be drawn around the identified object. The vector that defines the bounding box may include a set of pixels or other spatial coordinates that define the corners of the bounding box. By way of example, each pixel in the image may be represented by a pair of integers (such as an (x, y) pair) that index into a column number and a row number of the grid of pixels that comprises the image, respectively. These index numbers may be defined relative to an origin pixel (e.g., (0, 0)) defined as one of the corners of the image.

At step 406, the controller may rank the set of objects identified in step 404 into an ordered list. The objects may be ranked according to a default priority scheme specified by an administrator. Alternatively or additionally, when the user specifies which objects to track using the user interface described above, the user may also be able to manually specify the order or priority of the selected objects. Alternatively or additionally, the user may simply select one among a list of predefined priority schemes. The predefined priority schemes may be customized for and labelled according to patient status (e.g., critical, serious, stable, recovery, rehab, etc.), patient medical condition (e.g., heart attack, stroke, fall risk, pulmonary dysfunction, etc.), department (e.g., emergency department, intensive care unit, neonatal intensive care unit, medical/surgical ward, cardiology, pulmonology, oncology, etc.). In another embodiment, the system may infer a specific priority scheme based on any of the information discussed above that may be available through patient medical records, facility databases, an initial scan of what objects are present in the room, and/or other criteria.

At step 408, the controller refers to the ordered list and actuates the PTZ camera to target and capture a high-resolution image of the first object in the ordered list. The targeted orientation of the PTZ camera may be achieved by first establishing a mapping between the field of view of the wide angle camera and the field of view of the PTZ camera. By way of example, during a camera initialization procedure, the controller may register the pan and tilt angles where the PTZ camera is centered on each of the four corner pixels of the image from the wide angle camera. Once this mapping is established, the controller may read the coordinates that define the bounding box of the first object in the ordered list, compute pan and tilt angles to center the PTZ camera on the target bounding box and actuate the pan and tilt controls of the PTZ camera to center the bounding box in the field of view of the PTZ camera. In addition, the controller may use the coordinates that define the bounding box to compute a zoom factor for the PTZ camera that represents the maximum possible zoom factor that includes the entire target bounding box within the PTZ camera's field of view. The controller may then actuate the zoom controls of the PTZ camera to achieve the computed zoom factor. Once the PTZ camera has been configured with the computer pan, tilt, and zoom parameters, the controller may capture one or more high-resolution images of the target object.

After a high-resolution zoom image of the target object is obtained at step 408, the controller may employ computer vision techniques to extract status information from the zoom image. The controller may employ further object detection algorithms to extract information from the object in the zoom image. By way of example, the controller may employ text recognition to interpret alphanumeric displays on a patient vitals monitor.

In one embodiment, the controller may generate one or more API calls to a multi-modal LLM that prompt the LLM to extract various pieces of information from the image. The prompt generated by the controller may request the LLM to return the values in a specific format, such as JSON, CSV, or the like. In another embodiment, the controller may generate a prompt requesting the LLM to first return a list of information fields currently visible in the image. Subsequently, the controller may generate a prompt requesting the LLM to return the corresponding values currently displayed for all or a subset of the information fields visible in the image. The specific subset of information fields may be selected by a user in real-time or predefined in a configuration file.

For example, if the object detected is a patient vitals monitor, the controller may generate a text-based prompt requesting the LLM to return the values displayed in the image for pulse, oxygen saturation, systolic blood pressure, diastolic blood pressure, end-tidal carbon dioxide, respiration, oxygen saturation, and/or other medical information that may be displayed on the vitals monitor.

In another example, if the detected object is an intravenous fluid (IV) bag, the controller may generate a prompt requesting the LLM to return the name or type of the fluid and the amount of fluid remaining in the bag, which may be determined by comparing a visible line across the bag created by the surface of the fluid, the capacity of the fluid bag (often printed on the bag), and/or graduated volume markings that may be printed on the bag. The system may also be configured to capture high resolution images of the IV tubing, valves or stopcocks that may be connected to the tubing, as well as the IV port that connects the IV tubing to the IV line in the patient. With these images, the controller may prompt the LLM to return whether the IV bag is in fact connected to the patient and whether the fluid line is open or closed.

In yet another example, if the detected object is the patient's oxygen equipment, the controller may prompt the LLM to return the current mix of oxygen the patient is receiving. Where the controller detects an IV pump, the controller may prompt the LLM to return the name and dosage or rate of the medication being delivered by the pump.

If the controller detects a medical image display device in the room, the controller may prompt the LLM to describe the medical imagery being displayed on the device. For example, if the controller detects an x-ray image displayed on a device in the room, the controller may prompt the LLM to describe the content of the x-ray image. In this case, the LLM may analyze the x-ray and return a description of the x-ray to the controller (e.g., “the x-ray image shows the lumbar spine”).

In addition, the controller may also prompt the LLM to return other information about the room or the patient that may be apparent in the image. For example, the LLM may return an indication of whether the patient is in the room, a description of the patient's position in the room (standing, lying in bed on back, lying on side, prone in bed, sitting up in bed, sitting in chair, etc.), whether the door to the patient room or a bathroom in the patient room is open or closed, whether a television in the room is on, whether there is a visitor or staff member in the room, etc.

The controller may also include a microphone that can be used to analyze audio in the room. The controller may be configured to detect certain audio signatures, such as speech, coughing, sneezing, snoring, moaning, shouting, loud noises that may indicate a fall, or other sounds that may be useful in monitoring the patient and or staff that may be present in the patient room.

Each of the events or status determinations discussed above may be timestamped. The controller may use any time stamped values to generate plots, trendlines, activity histories, or other visual representations that may assist the care team in better understanding the patient's condition.

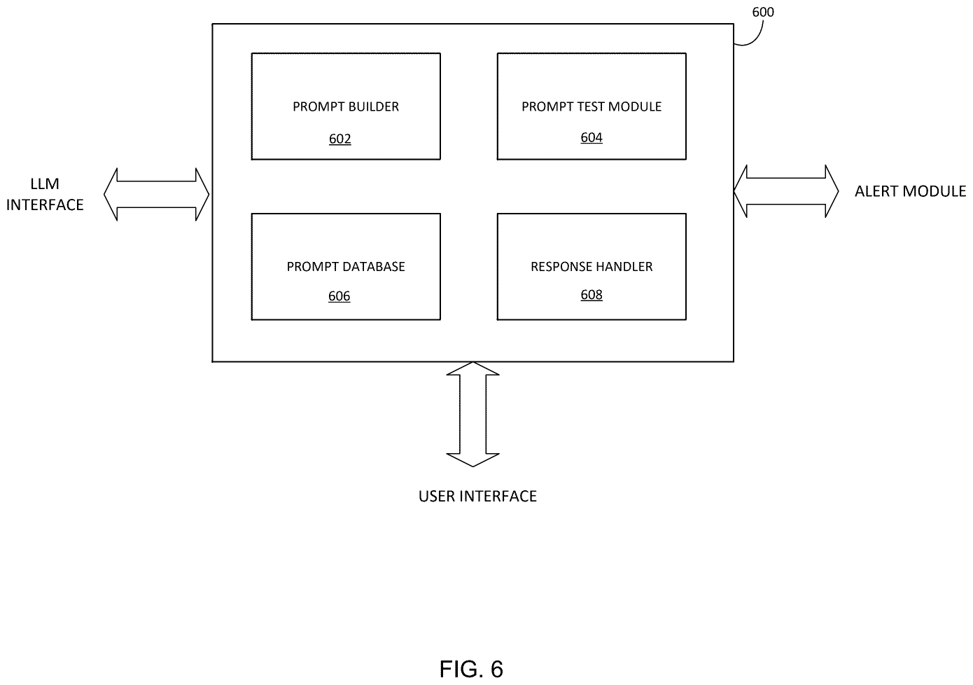

At step 412, the information extracted from the image at step 410 is displayed in a user interface or dashboard, such as that depicted in FIG. 6 and described more fully below. The fields contained within the dashboard may be configurable by the user and/or automatically arranged by the controller based on the information available from the images and/or other information sources.

The dashboard may be further configured with acceptable ranges for any of the information fields contained within it. If any of the status values fall outside their respective accepted ranges, the controller may generate an alert to at step 414.

At step 416, the controller removes the current or first object from the ordered list or otherwise advances an index to the next object in the list. The flow then proceeds to step 418, where the controller determines whether a wide angle image refresh timer has elapsed. The wide angle image refresh timer may be set to any time period suitable for keeping care staff updated on changes within the patient environment. For example, depending on the condition of the patient and/or the patient room, the wide angle refresh time may be set to anywhere from 1 second to several hours to several days. If the wide angle refresh timer has not expired, the controller proceeds to step 408 and repeats steps 402-416 for the next object in the ordered list. If, at step 418, the controller determines the wide angle refresh timer has expired, the controller proceeds to step 402 and repeats the entire process 400 as described above with a new wide angle image.

In addition to the wide angle refresh timer, the sequential targeting of the PTZ camera on each object in the ordered list may be slowed or otherwise controlled by a wait loop. This may be desirable where the patient is aware of their surroundings and finds continuous movement of the PTZ camera annoying or unsettling. By way of example, the controller may be configured to only target the PTZ camera on an object of interest every 10 minutes, or half-hour, or hour, etc.

The system may also leverage the MMLLM to improve the precision or granularity of the object detection/identification at step 404, which in turn can enhance the ability of the MLLM to extract relevant information about the identified objects at step 410, thereby improving the overall efficiency and functionality of the monitoring function. To illustrate, when the system performs object detection on the image at step 404, the object detection layer may detect several screens in the room but be unable to further distinguish among the identified screens (e.g., television, vitals monitor, computer monitor). To address this, the controller may prompt the LLM to further identify the identified objects in the image using additional contextual information. For example, the controller may prompt the LLM to identify the different types of screens in the image given the fact that the image was taken in a hospital room. With this additional context, the LLM may be able to distinguish among the screens to properly identify one screen as a television, another as a vitals monitor, and yet another as a computer monitor. The LLM may report this information back to the controller, which may then rely on these more accurate object labels to re-rank the objects at step 406 as well as inform further prompts to the LLM to extract additional information relating to the objects identified in each zoom image at step 410.

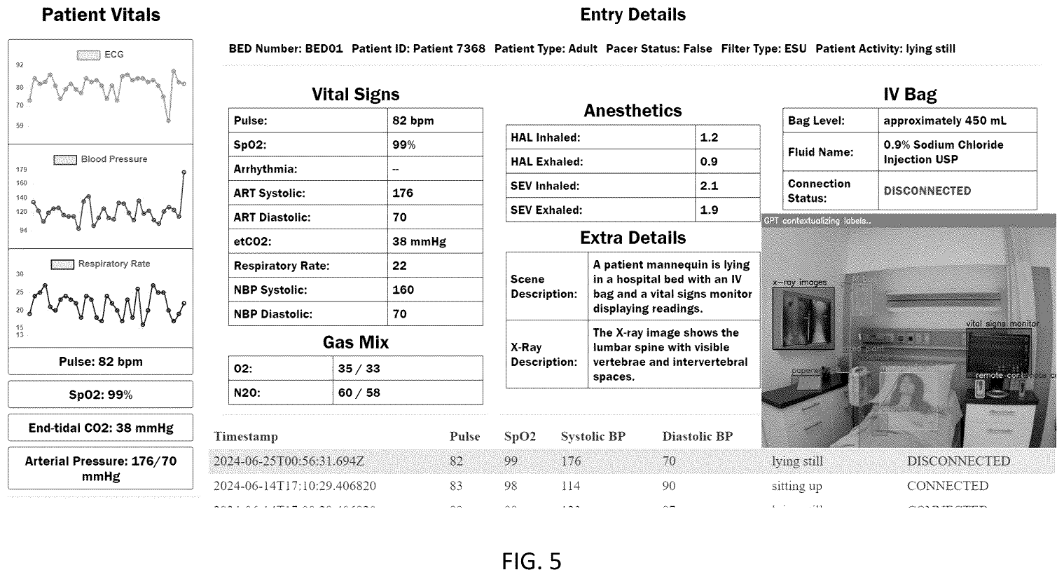

FIG. 5 illustrates an example user interface screen or “dashboard” that may be displayed to members of the patient's care team throughout the process described above and described more fully in U.S. provisional patent application No. 63/671,250, filed Jul. 14, 2024, the content of which is hereby incorporated by reference in its entirety. The fields displayed in user interface may be configured by the user or an administrator. Alternatively, the fields displayed may be selected automatically by the system based on the objects detected by the object detection system. By way of example, as shown in the image displayed in the lower right corner of FIG. 5, the object detection system has identified and drawn bounding boxes around, among other things, an x-ray image, an IV bag, a patient, an IV tube, and a vital signs monitor. Accordingly, the system may be configured to automatically display a field for each of these objects within the user interface.

It is to be appreciated that the above-described system and method described could also be achieved using a single, stationary camera with sufficient resolving power. In addition, though the above description focuses on a hospital room environment, the system and method described herein could also be deployed in homes and other locations where autonomous patient monitoring may be desired. In addition, instead of using an LLM, the system could be implemented solely with object detection algorithms. However, it should also be appreciated that when the autonomous patient monitoring system has access to a multi-media LLM, the MMLLM may provide the same functionality of and eliminate the need for the various detection modules 304, 306, 308 described with respect to FIG. 3. For example, rather than relying on a dedicated object detection algorithm, the system could alternatively prompt an MMLLM to detect and identify objects within the video, recognize voices, and/or interpret the outputs of connected vitals monitors, bed alarms, etc. Further, when the above-described system has access to an MMLLM, additional functionality can be achieved in the form of prompted event detection, as discussed more fully below.

FIG. 6 is a schematic diagram of a prompt manager 600, which may form part of an autonomous patient monitoring module with access to a multi-modal large language model, such as that discussed above with respect to FIG. 3. Alternatively, the prompt manager 600 may execute on any other device coupled to the above-described telehealth system via a network. The prompt manager 600 may include several software modules, including a prompt builder 602, a prompt test module 604, a prompt database 606, and a response handler 608.

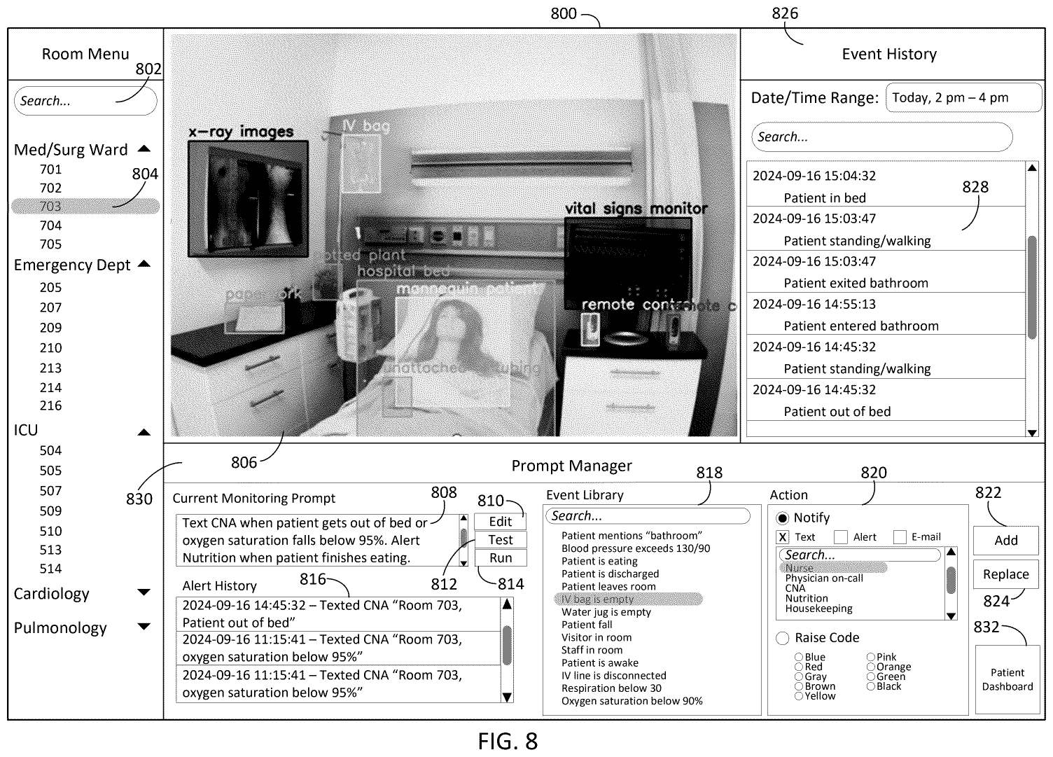

As discussed more fully below with respect to FIG. 8, the prompt builder 608 may be a text editing module configured to construct text-based prompts for use in autonomous patient monitoring using a multi-media LLM. The prompt builder 602 may receive text entered by a user via a keyboard or speech-to-text converter. In addition, the prompt builder may receive prompt text based on a use selection from prompt database 606, which stores predefined prompts that can be selected by a user via a user interface. The prompt builder can also receive prompt text from the response handler 608 when the handler determines further information is needed from the LLM. In any case, once the prompt has been constructed in the prompt builder, it can be communicated to the LLM to trigger a response from the LLM.

When a user enters a new prompt, they may wish to first test the prompt against available inputs to evaluate whether the prompt produces the desired response from the LLM. In this case, the new prompt text may be run through the prompt test module 604. The prompt test module 604 may run the prompt through the LLM with the currently available inputs and report any errors back to the user without updating any patient dashboards or generating alerts, etc. By way of example, where the user creates a prompt to “alert the nurse if the patient's heart rate exceeds 120 bpm,” but the system cannot, from available inputs, determine the patient's heart rate, the test module 604 may report this fact back to the user via the user interface. Similarly, where the user prompts the system to “text CNA when fluid bag is empty,” but the system cannot confirm the presence of a fluid bag, the test module 604 may report this to the user via the user interface.

In situations where the prompt being tested implicates elements not detectable at the time the prompt is tested, the user may choose to run the prompt anyway. For example, if the prompt being tested were, “count the number of times someone other than the patient enters and exits the room and send this count to ‘faculty_audit’ database every 2 hours,” the prompt test may notify the user that it “cannot currently detect ‘someone other than the patient’ in the room.” The user may then opt to run the prompt anyway on the assumption that the system will detect someone other than the patient when such a person enters the room.

In general, the test module 604 will verify that each element referenced in the prompt corresponds to an object that can be detected or recognized in the input video or is otherwise capable of being detected or tracked in the available input data. If not, the test module may report this to the user via the user interface module. The test module may also verify that any action specified in the prompt text corresponds to an action or service that the alert module has been configured to provide. If not, the test module may report this to the user via the user interface module.

The response handler 608 may be configured to interpret the LLM's responses to the prompt(s) and communicate with other elements of the system to perform the action specified in the prompt text when an event specified in the prompt text has been detected. For example, when the monitoring system transmits a snapshot of the a vitals monitor to the LLM and prompts the LLM to return a specific set of values in the image, the values returned by the LLM are received and handled by the response handler 608. The response handler may match data labels in the LLM's response to information fields in the patient record and/or dashboard and update those fields accordingly. In addition, when the prompt specifies an alert when an event is detected, and the response handler 608 receives a response from the LLM interface that the event has been detected, the response handler communicates with the alert module to raise or otherwise carry out the specified alert. As another example, if the monitoring system prompts the LLM to confirm that the patient is in bed, and the LLM cannot confirm that the patient is in bed, the response handler may update the patient bed status in the dashboard, which may then be used to raise an alert, if such an alert has been created for the subject patient. In yet another example, when the prompt specifies that certain activities detected in the video be logged in a database or activity log, the response handler 608 communicates with the appropriate database interface to ensure that the database or log is updated to reflected the detected activities and their respective timestamps.

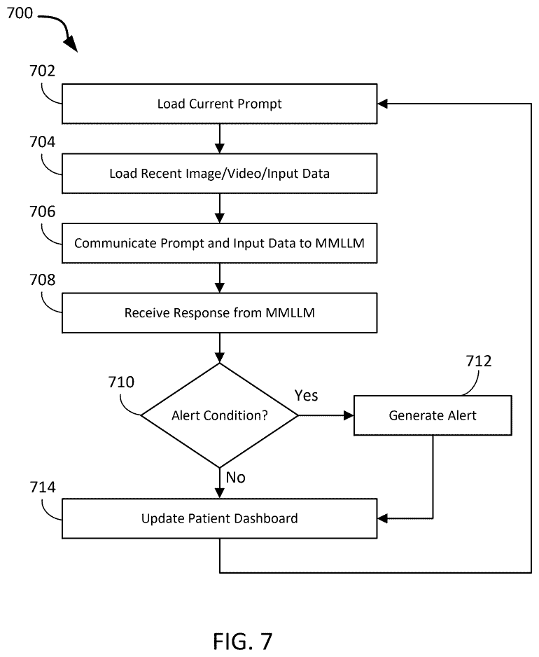

FIG. 7 is a flow chart illustrating a prompted event detection process 700 that may be performed by a controller or server in a telehealth system such as that described above. The process begins at step 702 when the controller loads the current or active prompt from the prompt manager. At step 704, the controller additionally loads recent image data, video data, input data from available devices, and/or contextual information relating to the input data and/or the prompt itself. At step 706, the controller communicates the prompt and the input data to the MMLLM via the LLM interface. At step 708, the controller receives a response from the LLM interface and parses the response data with the response handler.

At step 710, the controller determines whether the response data triggers an alert condition. If the response does trigger an alert condition, then the alert module will generate or raise the appropriate alert, such as a push notification in a mobile app to a particular staffer, SMS text message, or the like. If the controller determines at step 710 that no alert condition is present in the response data, the process proceeds to step 714, at which point the response data is used to update corresponding data fields in the patient's dashboard or other medical records. Whether or not an alert condition is detected at step 710 and an alert generated at step 712, the patient dashboard is updated at step 714. After the patient dashboard is updated at step 714, the process returns to step 702.

FIG. 8 illustrates an example user interface 800 for a prompted event detection system as described above. The interface includes a “Room Menu” bar on the left side of the screen that includes a list of locations that can be monitored using the prompted event detection system. In the current example, these locations would be hospital rooms that include in-room devices capable of supplying video and/or other inputs to the controller of the monitoring system. The Room Menu may include expandable/collapsible lists of rooms organized by departments or some other scheme. The Room Menu may also include a search box 802 where the user can input an alphanumeric string to filter the Room Menu to only those rooms whose names match or contain the search string. The Room Menu may also include a cursor 804 that allows the user to highlight and select a specific room for monitoring.

Once a room is selected with the cursor 802, the other windows of the user interface screen are populated with information associated with the selected room. For example, video from an in-room device at the selected room may be displayed in video window 806, the event history window 826 may display a series of events 812 detected in the selected room, and the prompt manager window 830 may display the current monitoring prompt 808 and alert history 816 for the selected room.

In addition to the current or active monitoring prompt 808 and alert history 816, the prompt manager window 830 may also include a number of tools that can be used to create, modify, test, and execute monitoring prompts. In general, the user can create or modify the current monitoring prompt either by clicking the edit button 810 and simply entering the desired prompt text or by using the event library 818 and actions 820 menus to construct the prompt text from lists of predefined events and actions that can be selected using those menus. When the user creates a prompt using the event library 818 and action menu 820, he or she may then select either the add button 822 to append the newly created prompt text to the current monitoring prompt text or select the replace button 824 to delete the current monitoring prompt text and replace it with the newly created prompt text. In the example illustrated in FIG. 8, the user has selected “IV bag is empty” from the event library 812 and selected to notify the patient's on-duty nurse via SMS text message. Once the user has made these selections, they may select the add button 822 or replace button 824.

Whether the user chooses to enter prompt text manually using the edit button 810 or use the events library 818 and actions menu 820, the user may also test the prompt text, as discussed above, by selecting the test button 812. When the user is satisfied with the text of the current monitoring prompt 808, he or she may activate the prompt by selecting the run button 814. Alternatively, the interface 800 may immediately execute whatever text is present in the current prompt box 808 and automatically test and report any errors encountered with the prompt text.

The action menu 820 includes a number of available actions to trigger when an alert condition is detected. The available actions may include a notification, which may be delivered via SMS text message, an “alert” or push notification sent within the monitoring software application itself or another, third-party application in communication with the monitoring system, and/or an email. The notification option includes a searchable list of potential recipients, or groups of recipients, that can be selected by the user to receive the notification.

The action menu 820 may also include an option to raise a hospital code when an alert condition is triggered. The menu includes a number of available hospital codes corresponding to different types of emergencies, which are known to those skilled in the art. Alternatively, the monitoring software may automatically determine the appropriate code to raise based on the description of the event that triggered the alert.

In general, the user may configure any number of actions to be triggered by a particular event. For example, either through the action menu 820 or directly editing the current monitoring prompt 808, the user may configure the system to “text CNA and alert Physician on-call when the patient is awake.”

The alert history window 816 may display a scrollable list of alerts that have been triggered for the currently selected room. Each item in the alert history window 816 may include a date and time stamp when the alert was triggered along with a verbal description of the action taken by the system in response to the alert condition being triggered.

The event history window 826 may display scrollable list of events the system has detected in the selected room or rooms. Each event 828 in the list may include a date and time stamp indicating when the event was detected and a verbal description of the detected event. An event 828 may also include visual indications of the detected event, such as graphical icons (bed, toilet, person standing, person on the ground, etc.) and/or color coding or other visual indications of the type of event detected. For example, detected events that pose a higher risk of fall or other danger to the patient (such as getting out of bed or walking around the room alone, detached IV line, etc.) may be highlighted in red, while events that indicate more routine intervention (such as the patient asking to go to the bathroom or an IV bag nearing empty) may be highlighted in yellow.

The events 828 displayed in the event history window 826 may not necessarily be alert events, such as those reported in alert history window 816. Rather, the event history window may simply display a log of every event tracked in the selected room. As shown in FIG. 8, the event history window 826 may also include a search bar that allows the user to filter the history of events displayed to only those containing user-specified text strings. The event history window 826 may also include a date and time range tool that allows the user to filter the history of events displayed to only those timestamped within a user specified date and/or time range.

The system may include a separate interface or window that allows the user to configure which events should be tracked in one, some, or all rooms in the telehealth system. The user may list or otherwise describe the events to be tracked in a monitoring prompt window similar to window 808. The tracked events may be written to a database that can serve as a general activity log for every room or care location that can be monitored within the telehealth system. This searchable activity log may be useful to auditors, facility managers, quality assurance managers, and the like. In addition, because the tracked events are stored in the form of text, the activity log represents a relatively small, low-cost storage solution for maintaining a history of events for even large facilities over long periods of time.

The user interface 800 may also include a patient dashboard button 832 that, when selected, displays the patient dashboard described above with respect to FIG. 5, which may itself include a button to return the user to the monitoring prompt interface 800.

Although the functions described above with respect to FIG. 8 are discussed in the context of a single selected patient room, the interface 800 may also include the ability to apply one or more prompts to multiple video feeds received from different patient rooms. For example, using the room menu, the user may manipulate an input device to select multiple rooms in the room menu. From there, the current monitoring prompt window 808 would display any monitoring prompts common to the multiple selected rooms and allow the user to add, edit, or otherwise modify the current monitoring prompt for the selected rooms. In another example, rather than individually selecting multiple rooms in the rooms menu, the user highlight an entire department or enter a range of room numbers in the room menu 802 search bar and then click a “select all” option in the filtered list of room numbers. In yet another embodiment, the user could simply include the desired room numbers, department(s), or range of room numbers in the text of the current monitoring prompt 808 to apply the current monitoring prompt to those rooms. When multiple rooms are selected in the room menu, the video window 806 may display video from each of the selected rooms in a split-screen video format.

Moreover, though the above description focuses primarily on clinical monitoring, it is to be appreciated that the described autonomous patient monitoring system could also monitor the operational status of a particular environment. By way of example, the system could be used to track and alert staff when garbage or empty food trays should be removed from a room, how frequently staff members check on the patient's condition, how recently the bed linens were changed, whether sanitation procedures are being followed, and/or tracking the movement of medical or other equipment in and out of a room. In addition, where speech detection and transcription are available, the system may also be useful for tracking staff compliance with patient evaluation and care protocols.

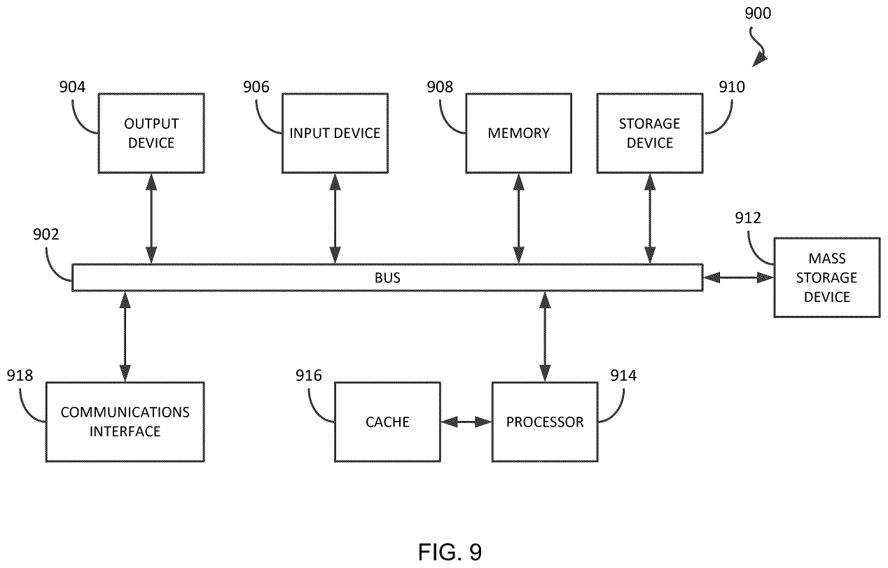

FIG. 9 depicts an example computing device or computer system 900 that may implement various systems and methods discussed herein. The computer system 900 includes one or more computing components in communication via a bus 902. In one implementation, the computer system 900 includes one or more processors 914. Each processor 914 may include one or more internal levels of cache 916, as well as bus controller or bus interface unit to direct interaction with a bus 902.

A memory 908 may include one or more memory cards and control circuits (not depicted), or other forms of removable memory, and may store various software applications including computer executable instructions, that when run on the processor 914, implement the methods and systems set out herein. Other forms of memory, such as a mass storage device 910, may also be included and accessible, by the processor (or processors) 914 via the bus 902.

The computer system 900 may further include a communications interface 918 by way of which the computer system 900 can connect to networks and receive data useful in executing the methods and system set out herein as well as transmitting information to other devices. The computer system 900 may include an output device 904, such as graphics card or other display interface by which information can be displayed on a computer monitor. The computer system 900 can also include an input device 906 by which information is input. Input device 906 can be a mouse, keyboard, scanner, and/or other input devices as will be apparent to a person of ordinary skill in the art.

The system set forth in FIG. 9 is but one possible example of a computer system that may employ or be configured in accordance with aspects of the present disclosure. It will be appreciated that other non-transitory tangible computer-readable storage media storing computer-executable instructions for implementing the presently disclosed technology on a computing system may be utilized.

The described disclosure may be provided as a computer program product, or software, that may include a computer-readable storage medium having stored thereon instructions, which may be used to program a computer system (or other electronic devices) to perform a process according to the present disclosure. A computer-readable storage medium includes any mechanism for storing information in a form (e.g., software, processing application) readable by a computer. The computer-readable storage medium may include, but is not limited to, optical storage medium (e.g., CD-ROM), magneto-optical storage medium, read only memory (ROM), random access memory (RAM), erasable programmable memory (e.g., EPROM and EEPROM), flash memory, or other types of medium suitable for storing electronic instructions.

The description above includes example systems, methods, techniques, instruction sequences, and/or computer program products that embody techniques of the present disclosure. However, it is understood that the described disclosure may be practiced without these specific details.

While the present disclosure has been described with references to various implementations, it will be understood that these implementations are illustrative and that the scope of the disclosure is not limited to them. Many variations, modifications, additions, and improvements are possible. More generally, implementations in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in the claims that follow.

What is claimed is:

1. A method for autonomous patient monitoring in a telehealth system, the method comprising:
displaying a user interface at a provider device, the user interface including a care location menu, a video window, and a prompt editor, the care location menu including a list including of a plurality of remote care locations;
receiving a selection of at least one care location from a user via a user input device;
displaying video received from a camera installed at the selected care location in the video window;
receiving a prompt via the prompt editor, the prompt comprising a text string that includes a description of an event relating to the care location and an action to be performed when the event is detected;
communicating an application programmer interface (API) call to a multi-media large language model (MMLLM) via an MMLLM interface, the API call including an instruction to notify a response handler when the event is detected in the video received from the care location;
receiving the notification from the MMLLM interface at the response handler; and
performing the action included in the prompt.

2. The method of claim 1, wherein the action includes transmitting an alert to a user device.

3. The method of claim 2, wherein the alert is transmitted to the user device using a short message service (SMS).

4. The method of claim 2, wherein the alert transmitted to the user includes a description of the event and the care location where the event was detected.

5. The method of claim 1, wherein the prompt editor displays a list of events and a list of actions and receiving the prompt includes receiving a selection of at least one event and at least one action from a user via the user input device.

6. The method of claim 1, wherein the event includes a patient getting out of bed.

7. The method of claim 1, wherein the event includes a patient's blood pressure exceeding a specified threshold.

8. The method of claim 1, wherein the user interface includes an event history window that displays a list of timestamped events detected in the video received from the care location.

9. The method of claim 1, wherein the prompt editor displays a notification when an element included in the prompt cannot be identified in the video received from the care location.

10. The method of claim 1, wherein the prompt includes a plurality of events and a corresponding plurality of actions to be performed when each respective event is detected.

11. A system for autonomous patient monitoring in a telehealth system, the system comprising:
a controller that displays a user interface at provider device, the user interface including a care location menu, a video window, and a prompt editor, the care location menu including a list of a plurality of remote care locations, wherein the controller is configured to:
receive a selection of at least one care location from a user via a user input device coupled to the provider device;
display video received from a camera installed at the selected care location in the video window;
receive a prompt via the prompt editor, the prompt comprising a text string that includes a description of an event relating to the care location and an action to be performed when the event is detected;
communicate an application programmer interface (API) call to a multi-media large language model (MMLLM) via an MMLLM interface, the API call including an instruction to notify a response handler when the event is detected in the video received from the care location;
receive the notification from the MMLLM interface at the response handler; and
perform the action included in the prompt.

12. The system of claim 11, wherein the action includes transmitting an alert to a user device.

13. The system of claim 12, wherein the alert is transmitted to the user device using a short message service (SMS).

14. The system of claim 12, wherein the alert transmitted to the user includes a description of the event and the care location where the event was detected.

15. The system of claim 11, wherein the prompt editor displays a list of events and a list of actions and receiving the prompt includes receiving a selection of at least one event and at least one action from a user via the user input device.

16. The system of claim 11, wherein the event includes a patient getting out of bed.

17. The system of claim 11, wherein the event includes a patient's blood pressure exceeding a specified threshold.

18. The system of claim 11, wherein the user interface includes an event history window that displays a list of timestamped events detected in the video received from the care location.

19. The system of claim 11, wherein the prompt editor displays a notification when an element included in the prompt cannot be identified in the video received from the care location.

20. The system of claim 11, wherein the prompt includes a plurality of events and a corresponding plurality of actions to be performed when each respective event is detected.
